(19) United States
(12) Patent Application Publication
(10) Pub. No.: US 2006/0235471 A1
(43) Pub. Date: Oct. 19, 2006

(54) Biological chip

(76) Inventor: Eukki Qi Yu, Industry, CA (US)

Correspondence Address: Raymond Y. Chan;Suite 128, 108 N. Ynez Ave., Monterey Park, CA, 91754, US

(21) Appl. No.: 11/107,693

(22) Filed: Apr. 14, 2005

(51) Int. Cl. A61N 1/00

(52) U.S. Cl. 607/2

(57) ABSTRACT

A biological chip provided for maintaining the health of a body cell through maintaining an original electrical signal expressed by the body cell, wherein the biological chip comprises a power source, a communication chip, and a biological carrier. The communication chip contains a correcting electrical signal, which is adapted for assisting the unhealthy body cell. The biological carrier forms a communication channel between the communication chip and the unhealthy body cell for transmitting the correcting electrical sign to the unhealthy body cell, so as to restore the unhealthy body cell back to the healthy condition. The correcting electrical signal corrects an unhealthy electrical signal transmitted by the cell without affecting healthy electrical signals transmitted by other healthy cells, such that the correcting electrical signal helps restoring the healthy condition of a cell, healing wounds, curing infections and converting harmful mutated cells to normal body cells, so as to cure diseases such as cancers and AIDS effectively. The biological chip is applied to a body in a transdermal manner such that no ingestion or injection of medication is required.

### BACKGROUND OF THE PRESENT INVENTION

1. Field of Invention

The present invention relates to a biological chip, and more particularly to a biological chip that maintain the healthy of body cells by conveying an electrical message to body cells, wherein the biological chip comprises a power source, a communication chip and a biological carrier. The communication chip contains a correcting electrical signal which is provided for correcting abnormal expressions of damaged or illness affected body cells and healing such cells, so as to cure disease and infections for conveying the electrical message to the body cells.

2. Description of Related Arts

It is common knowledge that human bodies are very weak and prone to diseases and illnesses, whether such diseases and illnesses are physical or metal. The reason is that the human body is constantly under the influence of different surrounding environment, and people.

Physical illnesses are caused by surrounding diseases or infection causing substances, such as free radicals, foreign viruses, pollutants, wherein such substances enters into the human body through surface wounds on the human body, inhalation and ingestion. Mental illnesses are caused by a weakness of mental health, which is also very much affected by the environment, such as sadness, stress and anxiety derived from work, relationship and their ambitions for achievement.

The reason why the human body is so prone to diseases and illnesses is that each human cell expresses a weak electrical signal which can easily be altered by other surrounding electrical signals. Originally, when a cell is healthy, it expresses a original electrical signal and such signal is less prone to be altered when the body is healthy.

However, when the human body is weak, such original electrical signals can easily be affected by surrounding electrical signals, such that the cell expresses an unhealthy electrical signal. It is when the original electrical signals of the cell are altered into the unhealthy electrical signal that the body starts to suffer from diseases and illnesses.

And, conventionally, when people fall ill or suffer from infections, they would pay their physicians a visit and obtain medications for inputting into their bodies, in the hope that their illnesses or infections would be cured by killing the illness or infections causing bacteria or viruses. Such medicines may be useful in killing bacteria and viruses, however, cannot help the person from feeling weak and fatigued even after the recovery. And while the body remains weak, they may easily catch another cold or other illness.

As for other illnesses or diseases such as cancer, complications brought about by immune system diseases, such as AIDS or lupus, or even metal illnesses, which are not caused by infectious bacteria or viruses, that are caused by mutations of body cells, weakness in immune system, or the weakness in mental health, they cannot be easily be cured by drugs.

As an example, leukemia is blood cancer which essentially is badly mutated blood cells appearing in the blood stream. By treating the disease, such blood cancer cells must be eliminated. Upon an early discovery of the cancer, elimination of cancer cells can be achieved through chemotherapy.

Unfortunately, due to the fact that such treatment cannot distinguish badly mutated cancer cells from other healthy cells, cancerous cells, as well as the healthy cells, will be destroyed, in such a manner that after going through chemotherapy, the blood circulation system of the patient will basically have no blood cells left, leaving the patient very vulnerable, prone to infections and to be under extensive care, until new, hopefully healthy, blood cells are generated.

However, since such treatment can only destroy cells, the underlying cause of bad cell mutations remained unknown and untreated. In the case where the patient suffer again from leukemia, the last resort for the patient would be to receive a bone marrow transplant, where the patient will receive a matching, healthy bone marrow from a donor, such that new blood, without cancerous cells, can be generated by such bone marrow, if the patient's body does not repel the foreign bone marrow.

Furthermore, since the underlying cause of bad cell mutations remained unknown and untreated, many cancer patients will probably find that they will suffer again from cancer later on in their life, same type or not.

Treatments of other diseases are similar, where only symptoms of the diseases are treated, leaving the true, underlying cause untreated, such that the patients will suffer from illnesses over and over again.

As a result, in order to prevent people from having illnesses and diseases, as well as to more appropriately help people to recover from illnesses, a better mode of illness treating product must be provided, such that a human body will be better protected from the surrounding environment, and not only the symptoms of diseases, but also the underlying illnesses causing problems, are treated.

### SUMMARY OF THE PRESENT INVENTION

A main object of the present invention is to provide a biological chip that maintains the health of a body cell by maintaining a cell expression of the body cell governing the well-doing of the body cell, wherein the biological chip comprising a power source, a communication chip, and a biological carrier electrically, wherein the communication chip contains a correcting electrical signal which is adapted for correcting the an abnormal cell expression, so as to cure diseases and infections, and protects a original electrical signal from being affected by surrounding electrical signals.

Another object of the present invention is to provide a biological chip, wherein the power source, the communication chip with the correcting electrical signal recorded thereon and the polymer forms an electrical circuit.

Another object of the present invention is to provide a biological chip, wherein the correcting signal recorded on the communication chip is customized according to the original electrical signals of the cell, which is prone to being affected by different diseases, virus or mental problems.

Another object of the present invention is to provide a biological chip, wherein the correcting signal is determined by balance comparison, wherein the unhealthy electrical signal of the body cell is compared with a controlled original electrical signal.

Another object of the present invention is to provide a biological chip, wherein the correcting signal is expressed to abnormal body cells that are disease affected or damaged cells, wherein the cell expressions of the cells are altered.

Another object of the present invention is to provide a biological chip, wherein the correcting signal is expressed only to abnormal body cells, such that normal body cells are not affected.

Another object of the present invention is to provide a biological chip, wherein the nano metal is one that would form a semiconductor or an insulator, but not a conductor, with the polymer conductor.

Another object of the present invention is to provide a biological chip, wherein the correcting electrical signal is positively charged, such that the electrical signal neutralizes the negative charge expressed by a wound, encouraging the healing of the wound.

Another object of the present invention is to provide a biological chip, wherein the correcting electrical signal is conveyed to the body cell in a transdermal manner, without the need of ingestion of medications or injection into bloodstream.

Accordingly, in order to accomplish the above objects, the present invention provides a biological chip for communicating with an unhealthy body cell of a user, comprising:
a power source;
a communication chip, which is electrically connected to the power source, wherein the communication chip contains a correcting electrical signal stored therein; and
a biological carrier, adapted for contacting with a skin of the user, comprising a predetermined amount of polymer conductor having a low electrical resistance and a predetermined amount of non-conducting nano metal mixed with the polymer conductor, wherein the biological carrier forms a communication channel between the communication chip and the unhealthy body cell for transmitting the correcting electrical signal from the communication chip to the unhealthy body cell such that the correcting electrical signal is adapted for assisting the unhealthy body cell to self-repair so as to restore the unhealthy body cell back to a healthy condition.

These and other objectives, features, and advantages of the present invention will become apparent from the following detailed description, the accompanying drawings, and the appended claims.

### BRIEF DESCRIPTION OF THE DRAWINGS

FIG. 1 illustrates a biological chip according to the preferred embodiment of the present invention.

FIG. 2 illustrates the biological chip being used on a body according to the above preferred embodiment of the present invention.

FIG. 3 is a flow chart illustrating an alternative embodiment of the present invention.

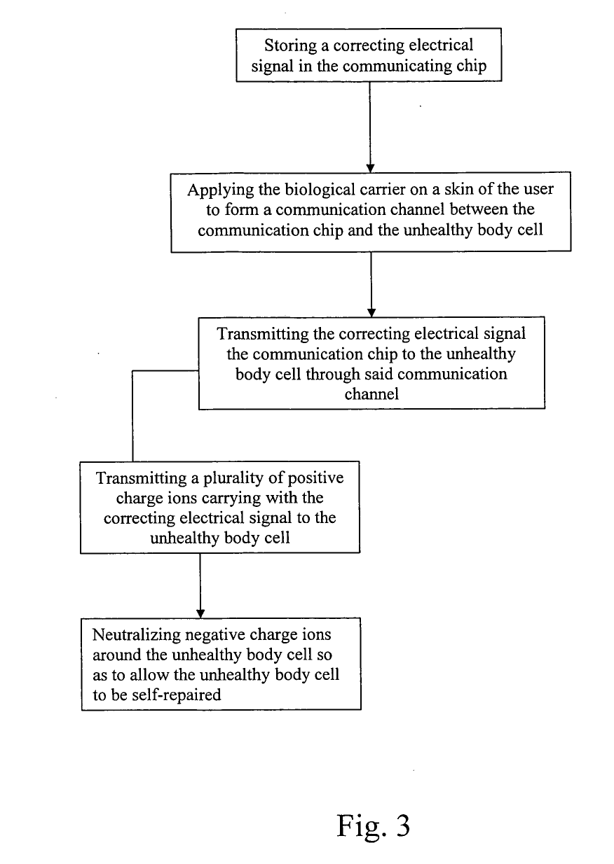

### DETAILED DESCRIPTION OF THE PREFERRED EMBODIMENT

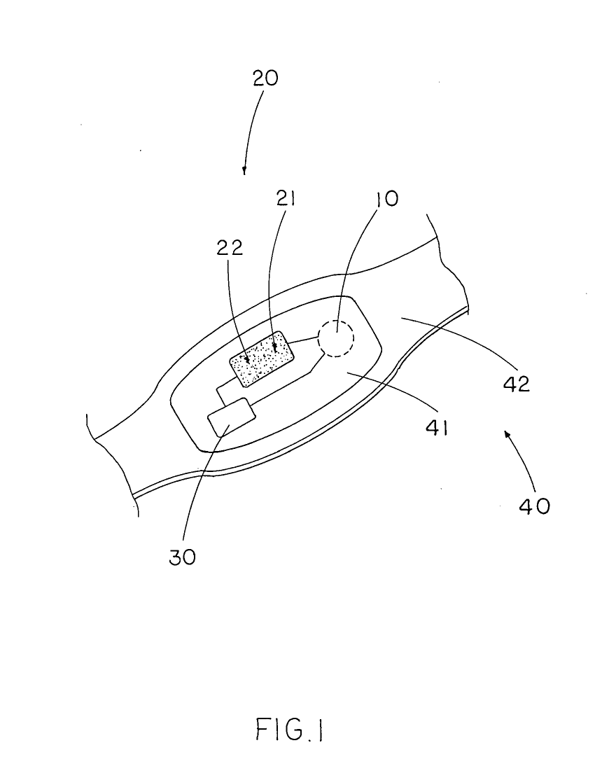

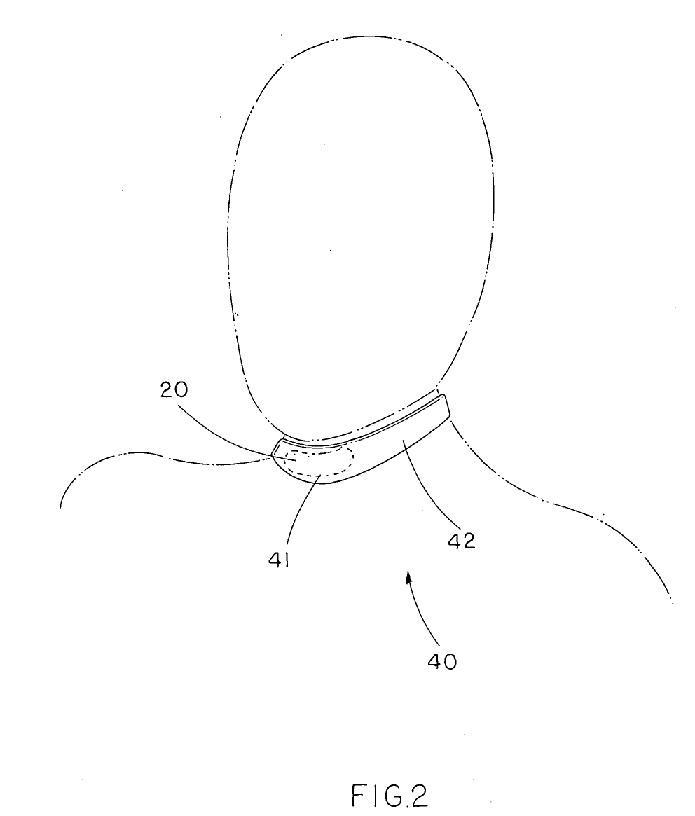

Referring to FIG. 1 and FIG. 2 of the drawings, a biological chip for communicating with an unhealthy body cell of a user according to a preferred embodiment of the present invention is illustrated, wherein the biological chip comprises a power source 10, a biological carrier 20, and a communication chip 30.

The power source 10 is provided for powering the biological chip through forming a complete circuit with the power source 10, the biological carrier 20, and the communication chip 30, wherein the communication chip 30 is electrically connected to the power source.

The biological carrier 20, adapted for contacting with a skin of the user, comprises a predetermined amount of polymer conductor 21 and a predetermined amount of nano metal 22, wherein the polymer conductor 21 and the nano metal 22 are thoroughly mixed together to form the biological carrier 20.

The power source 10, the biological carrier 20, and the communication chip 30 forms a closed electrical circuit. The communication chip 30 contains a correcting electrical signal stored therein.

The biological carrier 20 forms a communication channel between the communication chip 30 and the unhealthy body cell, such that the correcting electrical signal is transmitted from the communication chip 30 to the unhealthy body cell.

The polymer conductor 21 is a polymer which fully conducts electricity; the nano metal 22 does not conduct electricity but will form a semi-conductor with the polymer conductor 21 when mixed with the polymer conductor 21, which makes the biological carrier 20 a semiconductor that would conduct electricity only under certain conditions. According to the preferred embodiment of the present invention, the nano metal 22 is nano copper, nano gold or nano silver.

The correcting electrical signal is adapted for assisting the unhealthy body cell to self-repair so as to restore the unhealthy body cell back to a healthy condition. According to the preferred embodiment of the present invention, the electrical signal carriers a plurality of positive charged ions for transmitting to the unhealthy body cell to neutralize negative charged ions around the unhealthy body cell so as to allow the unhealthy body cell to be self-repaired.

The correcting electrical signal is pre-recorded in the communication chip 30 and is adapted to be customized for communicating with the unhealthy body cell at a particular tissue of the user.

The cell expression of a body cell is essentially an electrical signal expressed by a living body cell. Each living cell in a body has its own electrical signal to express. Such electrical signals are expressed for communication purposes with other cells, so as to keep the overall health of the body.

Originally when body is healthy and the cells are first produced, before any mutations or influence from the surrounding environment, a living cell would express an original electrical signal. However, when the cells are damaged, affected by viruses and bacteria, or under the influence of negative thoughts generated by the brain of the body, the cells would mutate and generate unhealthy electrical signals, which would be emitted to surrounding cells. That is when illness, mental or physical, results.

Conventionally, physical illnesses caused by bacteria or viruses are cured mostly by killing the disease causing bacteria or viruses, leaving the mutated cells to “heal” themselves, which would usually need a long time and may not be efficient. A lot of people have experienced the trouble of recurring illnesses, which is caused by unhealed mutated cells.

As for illnesses caused by the body itself, such as cancer, are cured by “killing” the cancerous cells, in the hope of getting rid of all the bad cells. However, as we all know, cancerous cells are very difficult to get rid of. And a person who has recovered from one type of cancer very often would find themselves suffering from another type of cancer later on in their lives.

The reason is that the unhealthy electrical signals remained in the body and continued to affect the cells. Furthermore, unhealthy electrical signals are also generated by negative thoughts of the brain of the body, which will continue to do so even after the cancerous cells are destroyed.

It is the reason why the unhealthy electrical signals must be corrected, so as to genuinely heal the damaged or mutated cells. By reversing the unhealthy electrical signals expressed by the abnormal body cells back to the original electrical signal, the abnormal body cells may truly heal and cease from affecting other healthy cells.

When the circuit formed by the power source 10, the biological carrier 20 and the communication chip 30 is closed, the electricity provided by the power source 10 initializes the communication chip 30 to transmit the correcting electrical signal recorded thereon.

The correcting electrical signal contains information that would correct the unhealthy signal expressed by the damaged, mutated, or disease or illness affected cells back to the original electrical signal, and protect a healthy signal expressed by a healthy cells from the influence of other unhealthy signals and surrounding environment.

According to the preferred embodiment of the present invention, the correcting electrical signal is customized according to how the were affected, by viruses or bacteria or by mental problems. In simpler terms, when trying to cure a particular disease, a corresponding correcting electrical signal would be recorded on the communication chip 30. And each disease is cured with a different correcting electrical signal.

When used to cure diseases not caused by bacteria or viruses, the correcting electrical signal is obtained by a technique called balance comparison where the unhealthy electrical signal is compared with a control original electrical signal.

When the communication chip 30 is electrically connected with the power 10 and the biological carrier 20, the communication chip 30 begins to express the correcting electrical signal in such a manner that the biological chip instructs body cells, and especially body cells that are illness affected or prone, to revert to a healthy state. The effect the biological chip has on the body cells is analogous to that of a CPU, which controls and instructs a machine to perform functions.

It is worth mentioning that the correcting electrical signal recorded on the communication chip affects only the unhealthy electrical signal of the illness affected cells. The correcting electrical signal will not affect the original electrical signal of healthy cells, but will provide the healthy cells with a protecting environment, where the healthy cells are surrounded by original electrical signals, preventing the healthy cells from being affected by any unhealthy signals around.

One advantage of forming a semi-conductor by the polymer conductor 21 and the nano metal 22 is that no complicated electrical circuitries or are required for the initializing of the biological chip such that it will not have circuitry problems and that it will have a better durability.

Another advantage of the present invention is that the original electrical signal the biological chip expresses is a small voltage positive signal. Wounds, infections and so forth are known to contain negative charges. Hence, by emitting the original electrical signal, the biological chip also helps to neutralize the negative charges of wounds and other infected tissues, speeding up the healing of such wounds and tissues.

It is also worth mentioning that the correcting electrical signal carries a weak positive voltage and therefore will not put the body under the threat of receiving of an electric shock. The weak positive voltage correcting electrical signal places damaged cells, as well as healthy cells, under a healthy environment of positive charge, as oppose to the negative charge emitted by damaged cells, which can control and eventually heal wounds and infections, and prevent infections caused by other negative unhealthy electrical signals of damaged cells.

Furthermore, since only weak voltage is required by the biological chip, the power source 10 can be any battery cells, disposable or rechargeable. According to the preferred embodiment of the present invention, the power source 10 is a button cell which is light, small and readily accessible, which enhances the usability of the biological chip.

Damaged Cells, as mentioned above, emits negative signals that would also affect healthy surrounding cells. The biological chip according to the present invention would mandatorily emit a positive correcting electrical signal that would, on one hand, suppress the negative unhealthy electrical signal emitted by the damaged cells and slowly correcting the electrical signal to the original electrical signal.

Once the damaged cells starts receiving the positive correcting electrical signal, they will also start accepting original electrical signals emitted by surrounding healthy cells, instead of affecting those healthy cells with their own unhealthy electrical signal, which would speed up the healing of the damaged cells.

On the other hand, the correcting electrical signal surrounds the healthy cells, acting as a shield for the healthy cells against the unhealthy electrical signal emitted from the damaged or mutated cells. This is particularly useful for people suffering from illnesses caused by their own immune system disorder such as AIDS, or other diseases cause by abnormal cell mutations, such as cancer.

The biological chip can effectively cure diseases transdermally without having to put the patient through medications or operations. It is applicable to all cells on the body, including organ tissues, such that patients, including those with chronic diseases or serious illness need not going through swallowing a consideration amount of pills or intravascular injections, which would lower the sufferings of patients.

It is especially advantages to patients with serious illnesses, such as cancer, which usually require operations that are costly yet may not be able to cure the diseases. The biological chip can cure such diseases without imposing high fees on the patient and prevent putting the patients under risks related to operations while they already are weak enough, and to prevent them from receiving any further suffering through unsuccessful surgeries.

Furthermore, most medications can cause side effects, sometimes even acute side effects, on the patients because of the fact that medications are essentially chemicals. Many chemicals used in medications, despite their useful aspects, are not naturally found in the body and could easily cause side effects. By the use of the present invention to cure diseases, the patient may effectively recover without being put under the risk of receiving side effects by taking conventional medications.

Referring to FIG. 2 of the drawings, according to the preferred embodiment of the present invention, the biological chip further comprises a wearing band 40. The wearing band 40 has a carrying pocket 41 receiving the power source 10, the communication chip 30 and the biological carrier therein. The wearing band 40 also has a wearing loop 42 extended from the carrying pocket for retaining the carrying pocket 41 on a body of the user at a position that the biological carrier 20 is in contact with the skin of the user.

Furthermore, the correcting electrical signal can be transmitted to the unhealthy body cell in a continuous manner or a periodical manner, depending on the need of the user and the severity of the illness or injury.

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A biological chip for communicating with an unhealthy body cell of a user, comprising:
a power source;
a communication chip, which is electrically connected to said power source, wherein said communication chip contains a correcting electrical signal stored therein; and
a biological carrier, adapted for contacting with a skin of said user, comprising a predetermined amount of polymer conductor having a low electrical resistance and a predetermined amount of non-conducting nano metal mixed with said polymer conductor, wherein said biological carrier forms a communication channel between said communication chip and said unhealthy body cell for transmitting said correcting electrical signal from said communication chip to said unhealthy body cell such that said correcting electrical signal is adapted for assisting said unhealthy body cell to self-repair so as to restore said unhealthy body cell back to a healthy condition.

2. The biological chip, as recited in claim 1, wherein said correcting electrical signal carries a plurality of positive charged ions for transmitting to said unhealthy body cell to neutralize negative charged ions around said unhealthy body cell so as to allow said unhealthy body cell to be self-repaired.

3. The biological chip, as recited in claim 2, wherein non-conducting nano metal is embodied as nano gold.

4. The biological chip, as recited in claim 2, wherein non-conducting nano metal is embodied as nano sliver.

5. The biological chip, as recited in claim 2, wherein non-conducting nano metal is embodied as nano copper.

6. The biological chip, as recited in claim 1, wherein said correcting electrical signal is pre-recorded in said communication chip and is adapted to be customized for communicating with said unhealthy body cell at a particular tissue of said user.

7. The biological chip, as recited in claim 3, wherein said correcting electrical signal is pre-recorded in said communication chip and is adapted to be customized for communicating with said unhealthy body cell at a particular tissue of said user.

8. The biological chip, as recited in claim 4, wherein said correcting electrical signal is pre-recorded in said communication chip and is adapted to be customized for communicating with said unhealthy body cell at a particular tissue of said user.

9. The biological chip, as recited in claim 5, wherein said correcting electrical signal is pre-recorded in said communication chip and is adapted to be customized for communicating with said unhealthy body cell at a particular tissue of said user.

10. The biological chip, as recited in claim 7, wherein said correcting electrical signal is penetrated transdermally through said skin of said user to said unhealthy body cell.

11. The biological chip, as recited in claim 8, wherein said correcting electrical signal is penetrated transdermally through said skin of said user to said unhealthy body cell.

12. The biological chip, as recited in claim 9, wherein said correcting electrical signal is penetrated transdermally through said skin of said user to said unhealthy body cell.

13. The biological chip, as recited in claim 10, further comprising a wearing band having a carrying pocket receiving said power source, said communication chip and said biological carrier therein, and a wearing loop extended from said carrying pocket for retaining said carrying pocket on a body of said user at a position that said biological carrier is in contact with said skin of said user.

14. The biological chip, as recited in claim 11, further comprising a wearing band having a carrying pocket receiving said power source, said communication chip and said biological carrier therein, and a wearing loop extended from said carrying pocket for retaining said carrying pocket on a body of said user at a position that said biological carrier is in contact with said skin of said user.

15. The biological chip, as recited in claim 12, further comprising a wearing band having a carrying pocket receiving said power source, said communication chip and said biological carrier therein, and a wearing loop extended from said carrying pocket for retaining said carrying pocket on a body of said user at a position that said biological carrier is in contact with said skin of said user.

16. A method of communicating with an unhealthy body cell of a user with a biological chip which comprises a communication chip and a biological carrier, comprising the steps of:
(a) storing a correcting electrical signal in said communicating chip;
(b) applying said biological carrier on a skin of said user to form a communication channel between said communication chip and said unhealthy body cell, wherein said biological carrier comprises a predetermined amount of polymer conductor having a low electrical resistance and a predetermined amount of non-conducting nano metal mixed with said polymer conductor; and
(c) transmitting said correcting electrical signal said communication chip to said unhealthy body cell through said communication channel, wherein said correcting electrical signal is penetrated transdermally through said skin of said user to said unhealthy body cell, such that said correcting electrical signal is adapted for assisting said unhealthy body cell to self-repair so as to restore said unhealthy body cell back to a healthy condition.

17. The method as recited in claim 16, in step (c), further comprising the steps of:
(c.1) transmitting a plurality of positive charge ions carrying with said correcting electrical signal to said unhealthy body cell; and
(c.2) neutralizing negative charge ions around said unhealthy body cell so as to allow said unhealthy body cell to be self-repaired.

18. The method, as recited in claim 17, wherein non-conducting nano metal is embodied as nano gold.

19. The method, as recited in claim 17, wherein non-conducting nano metal is embodied as nano sliver.

20. The method, as recited in claim 17, wherein non-conducting nano metal is embodied as nano copper.
